(19) United States
(12) Patent Application Publication
(10) Pub. No.: US 2025/0029708 A1
(43) Pub. Date: Jan. 23, 2025

(54) IMAGE CUTOUT SUPPORT APPARATUS, ULTRASOUND DIAGNOSTIC APPARATUS, AND IMAGE CUTOUT SUPPORT METHOD

(71) Applicant: FUJIFILM Corporation, Tokyo (JP)

(72) Inventor: Riki IGARASHI, Kanagawa (JP)

(73) Assignee: FUJIFILM Corporation, Tokyo (JP)

(21) Appl. No.: 18/907,452

(22) Filed: Oct. 4, 2024

Related U.S. Application Data
(63) Continuation of application No. PCT/JP2023/013111, filed on Mar. 30, 2023.

(30) Foreign Application Priority Data
May 26, 2022 (JP) 2022-086182

(51) Int. Cl. G16H 30/20; G06T 7/00

(52) U.S. Cl. CPC G16H 30/20; G06T 7/0012; G06T 2207/10132; G06T 2207/30004

(57) ABSTRACT

There is provided an image cutout support apparatus for cutting out a typical image including an object to be diagnosed by a user from video image data (M). The image cutout support apparatus includes a video image data input unit (11) that inputs the video image data (M), a screening unit (12) that performs image analysis on the video image data (M) to select a candidate image group related to the typical image from the video image data (M), and a recommendation unit (13) that gives a priority to each image of the candidate image group on the basis of at least one of the image analysis performed by the screening unit (12) or information of the user.

### CROSS-REFERENCE TO RELATED APPLICATIONS

This application is a Continuation of PCT International Application No. PCT/JP2023/013111 filed on Mar. 30, 2023, which claims priority under 35 U.S.C. § 119 (a) to Japanese Patent Application No. 2022-086182 filed on May 26, 2022. The above applications are hereby expressly incorporated by reference, in their entirety, into the present application.

### BACKGROUND OF THE INVENTION

### 1. Field of the Invention

The present invention relates to an image cutout support apparatus, an ultrasound diagnostic apparatus, and an image cutout support method that are used to cut out an image from video image data.

### 2. Description of the Related Art

In the related art, a diagnostic apparatus that captures an image of the inside of a subject indicating a tomographic image of the subject, such as a so-called ultrasound diagnostic apparatus, is known. In this diagnostic apparatus, video image data indicating the tomographic image of the subject may be obtained by continuously acquiring images of a plurality of frames indicating the tomographic images of the subject. In many cases, a user, such as a doctor, checks the video image data acquired by the diagnostic apparatus to diagnose the subject. In many cases, the diagnosis result obtained in this way is described in a report for recording or is shared with other users, such as doctors, for the treatment of the subject.

Here, in some cases, the user uses an image that well represents findings, such as diseases, of the subject, that is, an image that enables the user to relatively easily determine the findings of the subject to create a report or to share information with other users. Therefore, for example, JP2014-124235A discloses a technique for easily obtaining an image that well represents findings of a subject. JP2014-124235A discloses a configuration that detects the movement of an anatomical structure included in an image of an image group including the image of one frame designated by a user among images of a plurality of frames constituting video image data and averages the image group in consideration of the detection result to acquire a high-quality image.

### SUMMARY OF THE INVENTION

Here, in the technique disclosed in JP2014-124235A, it is necessary for the user to manually select an image of one frame among the images of the plurality of frames constituting the video image data. In this case, in particular, in order to acquire an image with higher quality, the user needs to check the images of the plurality of frames constituting the video image data and then to select an image of one frame that well represents the findings of the subject. However, this operation is usually complicated and requires a lot of effort from the user.

The present invention has been made to solve these problems of the related art, and an object of the present invention is to provide an image cutout support apparatus, an ultrasound diagnostic apparatus, and an image cutout support method that enable a user to easily select an image from video image data.

According to the following configuration, the above object can be achieved.
[1] There is provided an image cutout support apparatus for cutting out a typical image including an object to be diagnosed by a user from video image data, the image cutout support apparatus comprising: a video image data input unit that inputs the video image data; a screening unit that performs image analysis on the video image data to select a candidate image group related to the typical image from the video image data; and a recommendation unit that gives a priority to each image of the candidate image group on the basis of at least one of the image analysis performed by the screening unit or information of the user.
[2] In the image cutout support apparatus according to [1], the screening unit extracts images from the video image data for every predetermined number of frames.
[3] In the image cutout support apparatus according to [1], the screening unit excludes similar images located in neighborhood frames from an object to be selected as the candidate image group.
[4] In the image cutout support apparatus according to [1], the screening unit excludes an image indicating an aerial emission state from an object to be selected as the candidate image group.
[5] In the image cutout support apparatus according to [1], the screening unit excludes an image having a quality equal to or less than a predetermined threshold value from an object to be selected as the candidate image group.
[6] In the image cutout support apparatus according to any one of [1] to [5], the recommendation unit performs organ determination on each image of the candidate image group and gives a high priority to an image including an organ.
[7] In the image cutout support apparatus according to [6], the recommendation unit gives a higher priority to an image, in which the organ determined by the organ determination is related to the information of the user, in consideration of the information of the user.
[8] In the image cutout support apparatus according to any one of [1] to [5], the recommendation unit gives a high priority to an image, in which a preference of the user has been reflected, on the basis of the information of the user.
[9] The image cutout support apparatus according to [1] further comprises an image processing unit that performs image processing according to a preference of the user on the basis of the information of the user.

There is provided an ultrasound diagnostic apparatus comprising: an ultrasound probe; an image generation unit that transmits and receives an ultrasound beam to and from a subject using the ultrasound probe to generate video image data; and the image cutout support apparatus according to [1], in which the video image data generated by the image generation unit is input to the video image data input unit.

There is provided an image cutout support method for cutting out a typical image including an object to be diagnosed by a user from video image data, the image cutout support method comprising: inputting the video image data; performing image analysis on the video image data to select a candidate image group related to the typical image from the video image data; and giving a priority to each image of the candidate image group on the basis of at least one of the image analysis or information of the user.

According to the present invention, an image cutout support apparatus for cutting out a typical image including an object to be diagnosed by a user from video image data comprises a video image data input unit that inputs the video image data, a screening unit that performs image analysis on the video image data to select a candidate image group related to the typical image from the video image data, and a recommendation unit that gives a priority to each image of the candidate image group on the basis of at least one of the image analysis performed by the screening unit or information of the user. Therefore, the user can easily select an image from the video image data.

### BRIEF DESCRIPTION OF THE DRAWINGS

FIG. 1 is a block diagram illustrating a configuration of an image cutout support apparatus according to Embodiment 1 of the present invention.

FIG. 2 is a diagram schematically illustrating a display example of a candidate image group.

FIG. 3 is a flowchart illustrating an operation of the image cutout support apparatus according to Embodiment 1 of the present invention.

FIG. 4 is a block diagram illustrating a configuration of an ultrasound diagnostic apparatus according to Embodiment 2 of the present invention.

FIG. 5 is a block diagram illustrating a configuration of a transmitting and receiving circuit according to Embodiment 2 of the present invention.

FIG. 6 is a block diagram illustrating a configuration of an image generation unit according to Embodiment 2 of the present invention.

FIG. 7 is a block diagram illustrating a configuration of an image cutout support apparatus according to Embodiment 3 of the present invention.

### DESCRIPTION OF THE PREFERRED EMBODIMENTS

Hereinafter, embodiments of the present invention will be described with reference to the accompanying drawings.

The following description of components is based on a representative embodiment of the present invention. However, the present invention is not limited to the embodiment.

In addition, in the present specification, a numerical range represented by “to” means a range including numerical values described before and after “to” as a lower limit value and an upper limit value.

In the present specification, the terms “same” and “identical” include an error range generally allowed in the technical field.

### Embodiment 1

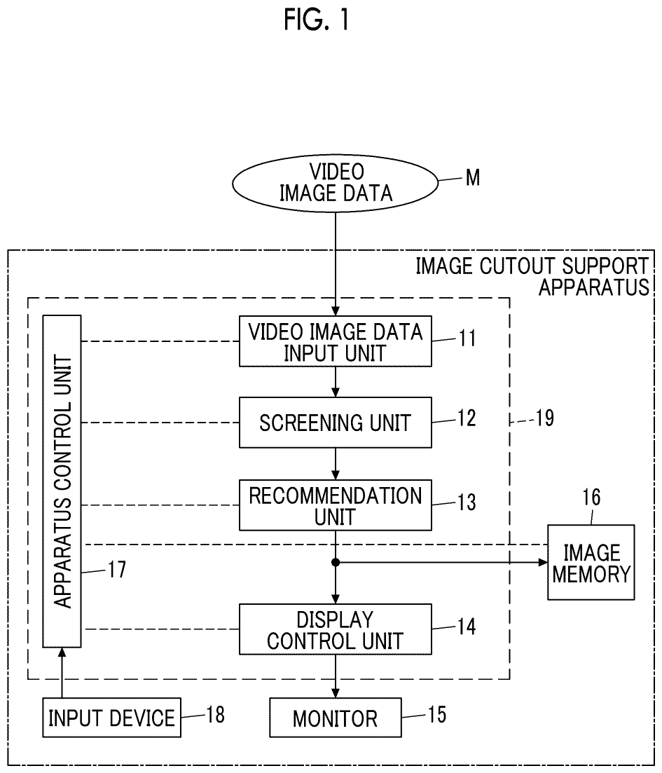

FIG. 1 illustrates a configuration of an image cutout support apparatus according to Embodiment 1 of the present invention. The image cutout support apparatus is connected to an external apparatus (not illustrated) of which examples include an apparatus that handles images or video images, such as a so-called ultrasound diagnostic apparatus or a so-called endoscope apparatus, and an apparatus that handles a series of images, such as a computed tomography (CT) apparatus, and acquires, from the apparatus, video image data M that indicates tomographic images of the inside of the subject and is composed of images of a plurality of consecutive frames. For example, in addition to the diagnostic apparatus, such as an ultrasound diagnostic apparatus, an endoscope apparatus, or a computed tomography apparatus, a so-called ultrasound probe can be connected to the image cutout support apparatus. In addition, a recording medium in which the video image data M is stored can be connected to the image cutout support apparatus.

The image cutout support apparatus has a video image data input unit 11 that is connected to an external apparatus (not illustrated) and inputs the video image data M from the external apparatus. A screening unit 12, a recommendation unit 13, a display control unit 14, and a monitor 15 are sequentially connected to the video image data input unit 11. In addition, an image memory 16 is connected to the recommendation unit 13. Further, an apparatus control unit 17 is connected to the video image data input unit 11, the screening unit 12, the recommendation unit 13, the display control unit 14, and the image memory 16. Furthermore, an input device 18 is connected to the apparatus control unit 17.

Meanwhile, in some cases, a user, such as a doctor, uses an image that well represents an anatomical structure or findings, such as diseases, of a subject, that is, an image that enables the user to relatively easily determine the anatomical structure or the findings, such as diseases, of the subject to create a report or to share information with other users.

The image cutout support apparatus according to Embodiment 1 of the present invention is an apparatus that supports the user in selecting and cutting out the image that well represents the anatomical structure or the findings, such as diseases, of the subject from images of a plurality of frames constituting the video image data M.

In addition, a processor 19 for an image cutout support apparatus is configured by the video image data input unit 11, the screening unit 12, the recommendation unit 13, the display control unit 14, and the apparatus control unit 17.

The video image data input unit 11 inputs the video image data M from an external apparatus (not illustrated) or the like. The video image data input unit 11 includes, for example, a connection terminal for wired connection via a communication cable or the like (not illustrated) to an external apparatus, such as a diagnostic apparatus or a recording medium, or an antenna or the like for wireless connection to the external apparatus.

A recording medium, such as a flash memory, a hard disk drive (HDD), a solid state drive (SSD), a flexible disk (FD), a magneto-optical disk (MO), a magnetic tape (MT), a random access memory (RAM), a compact disc (CD), a digital versatile disc (DVD), a secure digital card (SD card), or a universal serial bus memory (USB memory), is given as an example of the recording media connected to the video image data input unit 11.

The screening unit 12 performs image analysis on the video image data M input to the video image data input unit 11 to select a candidate image group related to a typical image from the images of a plurality of frames constituting the video image data M. The typical image is an image including an object to be diagnosed by the user, for example, the anatomical structure or the findings, such as diseases, of the subject to be diagnosed from the image. In addition, the candidate image group related to the typical image means an image group that is selected by the user, such as the doctor, as a candidate for an image that well represents the typical image, that is, an image that enables the user to easily determine the anatomical structure or the findings, such as diseases, of the object to be diagnosed.

For example, the screening unit 12 can extract images for every predetermined number of frames from the video image data M to select the candidate image group. In general, a time interval in a case where images of a plurality of consecutive frames representing the anatomical structure of the subject, such as so-called ultrasound images, are captured is often very short. Therefore, the images of a plurality of frames captured within a certain period of time are often similar to each other. The screening unit 12 extracts images from the video image data M for every predetermined number of frames and selects the candidate image group. In this way, the screening unit 12 can exclude images that are similar to each other from the object to be selected and select images having a low degree of similarity as the candidate image group.

In addition, the screening unit 12 can also perform a process of calculating the degree of similarity for an image of any one frame or a process of calculating the degree of similarity between images of a plurality of frames, for example, a general process, such as comparison of histograms, normalized cross-correlation processing, feature point matching, or comparison of embedding vectors of images by any learning model, on the images of the plurality of frames constituting the video image data M to exclude similar images located in neighborhood frames from the object to be selected. Therefore, the screening unit 12 can select the images having a low degree of similarity to each other as the candidate image group. In addition, the images of the neighborhood frames mean images of a plurality of frames captured in the same time range.

Meanwhile, in the capture of an ultrasound image by a so-called ultrasound probe, in a case where the ultrasound probe is separated from a body surface of the subject, ultrasound waves are emitted to the air from the ultrasound probe, and it is not possible to receive the ultrasound waves. As a result, for example, an image that is completely painted black is obtained. The image that is completely painted black in a so-called aerial emission state is generally not used to diagnose the subject. Therefore, the screening unit 12 can perform so-called histogram analysis or the like on the images of the plurality of frames constituting the video image data M to specify an image in the aerial emission state and exclude the specified image in the aerial emission state from the object to be selected as the candidate image group.

In addition, in a case where an ultrasound image indicating a tomographic image of the subject is captured by the ultrasound probe, a blurred ultrasound image, that is, an unclear ultrasound image with a low contrast may be obtained due to a speed at which the user moves the ultrasound probe on the body surface of the subject or the movement of the anatomical structure in the subject. Therefore, the screening unit 12 has a predetermined threshold value related to the quality of the image, can perform a process, such as so-called edge detection, on the images of the plurality of frames constituting the video image data M to calculate the quality of the ultrasound images of a plurality of frames, and can select an image having quality equal to or less than the threshold value as the object to be selected as the candidate image group. The quality of the image described herein means an indicator representing the sharpness of the edge of the anatomical structure of the subject included in the image.

The recommendation unit 13 gives a priority to each image of the candidate image group selected by the screening unit 12 on the basis of the image analysis performed by the screening unit 12, the information of the user, and the like. Here, the information of the user includes, for example, the type of medical department to which the user, who is the doctor, belongs, the type of anatomical structure selected by the user in the past, and the like. The information of the user can be input in advance, for example, through the input device 18.

For example, the recommendation unit 13 can perform organ determination on each image of the candidate image group and give a high priority to the image including the organ. In this case, for example, the recommendation unit 13 has template data indicating a typical shape and the like for each of a plurality of organs of the subject and can perform the organ determination using a so-called template matching method that compares the anatomical structure included in the image with a plurality of template data items.

The recommendation unit 13 has, for example, a learning model that has learned the shapes of the organs and the like included in the images using a model according to an algorithm, such as so-called Residual Neural Network (ResNet), Dense Convolutional Network (DenseNet), AlexNet, baseline, batch normalization, dropout regularization, NetWidth search, or NetDepth search, and can input the image to the learning model to perform the organ determination.

In addition, the recommendation unit 13 can also give a high priority to an image, in which the organ determined by the organ determination is related to the information of the user, in consideration of the information of the user. For example, a high priority can be given to an image including the organ, such as the bladder, related to a urology department in the candidate image group selected by the screening unit 12 on the basis of the information of the user who is the doctor and belongs to the urology department.

In addition, the recommendation unit 13 can also give a high priority to an image, in which the preference of the user has been reflected, on the basis of the information of the user. For example, the recommendation unit 13 can identify the user using an identifier (ID) or the like input by the user through the input device 18 and give a high priority to the image including the organ with reference to the information of the user indicating the type of the organ included in the image selected by the same user in the past. Here, the recommendation unit 13 has a threshold value determined for the number of times the same user selected the image including the same organ in the past and can also give a high priority to the image including the organ of which the number of selections in the past is equal to or greater than the threshold value.

Further, the recommendation unit 13 can also give a high priority to the image in which the organ determined by the organ determination is related to the information of the diagnostic apparatus that has captured the video image data M. For example, in a case where the diagnostic apparatus that has captured the video image data M is used in the urology department and information indicating that the diagnostic apparatus is used in the urology department is added to the captured video image data M, the recommendation unit 13 can give a high priority to the image including the organ, such as the bladder, related to the urology department in the candidate image group selected by the screening unit 12 on the basis of apparatus information indicating that the diagnostic apparatus is used in the urology department. Here, the apparatus information is linked to the images of the plurality of frames constituting the video image data M, for example, according to a standard such as so-called Digital Imaging and Communications in Medicine (DICOM).

The recommendation unit 13 can give the priority to the image on the basis of at least one of a plurality of conditions, such as the presence or absence of the organ in the image, the relation between the organ determined by the organ determination and the information of the user, the preference of the user, and the relation between the organ determined by the organ determination and the information of the diagnostic apparatus.

Further, the recommendation unit 13 can also give the priority to the image in consideration of the plurality of conditions, such as the presence or absence of the organ in the image, the relation between the organ determined by the organ determination and the information of the user, the preference of the user, and the relation between the organ determined by the organ determination and the information of the diagnostic apparatus, using an algorithm as so-called emphasis filtering.

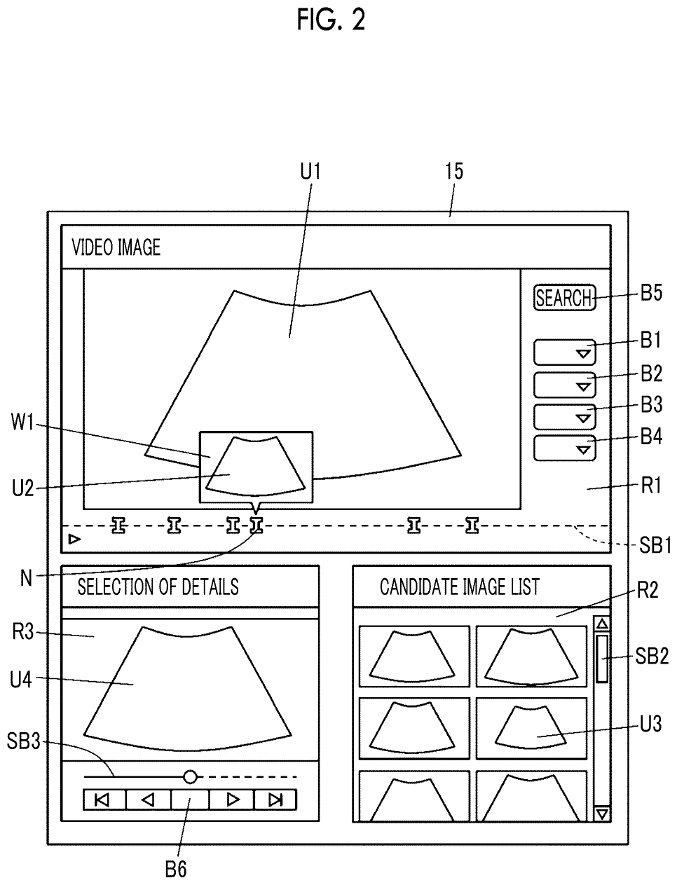

The apparatus control unit 17 controls each unit of the image cutout support apparatus according to a program or the like recorded in advance. In addition, the apparatus control unit 17 can display, for example, the image to which the priority has been given by the recommendation unit 13 and the images of the plurality of frames constituting the video image data M input by the video image data input unit 11 on the monitor 15 as illustrated in FIG. 2. In the example illustrated in FIG. 2, a first display region R1 displayed as “Video Image”, a second display region R2 displayed as “Candidate Image List”, and a third display region R3 displayed as “Selection of Details” are displayed on the monitor 15.

In the first display region R1, the following are displayed: condition selection buttons B1 to B4 for selecting the plurality of conditions, such as the presence or absence of the organ in the image, the relation between the organ determined by the organ determination and the information of the user, the preference of the user, and the relation between the organ determined by the organ determination and the information of the diagnostic apparatus; a search button B5 for giving a priority to each image of the candidate image group on the basis of the conditions selected by the condition selection button B1 to B4 and selecting an image to which a priority greater than a predetermined value is given; and a so-called slide bar SB1 for displaying each image of the candidate image group. In addition, a plurality of markers N that indicates the positions of the images of a plurality of frames selected by the selection of the search button B5 on a time axis to be emphasized are displayed on the slide bar SB1. For example, an image U1 corresponding to a position on the slide bar SB1 is displayed by operating the slide bar SB1 through the input device 18. In addition, for example, in a case where any of the plurality of markers N is selected, the image U1 corresponding to the marker N is displayed. Further, in a case where various buttons are selected by a so-called cursor, for example, a display window W1 including a minified image U2 can be displayed by placing the cursor on any of the plurality of markers N, which is not illustrated.

The user can check the first display region R1 to easily ascertain and select the image that well represents the anatomical structure or the findings of the subject in the candidate image group.

A plurality of images U3 to which a high priority has been given by the recommendation unit 13 and which have been minified and a slide bar SB2 are displayed in the second display region R2. The user can operate the slide bar SB2 to view the plurality of images U3 while sliding the plurality of images U3 in the vertical direction. The user can also check the second display region R2 to easily ascertain the image that well represents the anatomical structure or the findings of the subject.

In the third display region R3, the following are displayed: a slide bar SB3 for the user to view the images of the plurality of frames constituting the video image data M; an image U4 corresponding to an operation position of the slide bar SB3; and a selection button B6 for selecting the displayed image U4 as an image to be used for creating a report, sharing with other users, or the like. The user can manually select an image using the third display region R3.

Further, FIG. 2 illustrates an example in which the images U1 to U4 are ultrasound images.

The image memory 16 stores the image to which the high priority has been given by the recommendation unit 13. The user, such as the doctor, can easily select the image to be used for creating a report or sharing with other users from the image of at least one frame stored in the image memory 16. In addition, for example, a recording medium, such as a flash memory, an HDD, an SSD, an FD, an MO disk, an MT, a RAM, a CD, a DVD, an SD card, or a USB memory, can be used as the image memory 16.

The display control unit 14 performs predetermined processing on, for example, the images of the plurality of frames constituting the video image data M and displays the processed images on the monitor 15 under the control of the apparatus control unit 17.

The monitor 15 performs various types of display under the control of the apparatus control unit 17. For example, the monitor 15 can include a display device such as a liquid crystal display (LCD) or an organic electroluminescence (EL) display.

The input device 18 receives an input operation of the user and transmits input information to the apparatus control unit 17. For example, the input device 18 is configured by devices, such as a keyboard, a mouse, a track ball, a touch pad, and a touch panel, for an examiner to perform an input operation.

In addition, the processor 19 including the video image data input unit 11, the screening unit 12, the recommendation unit 13, the display control unit 14, and the apparatus control unit 17 of the image cutout support apparatus may be configured by a central processing unit (CPU) and a control program for causing the CPU to perform various processes. However, the processor 19 may be configured by a field programmable gate array (FPGA), a digital signal processor (DSP), an application specific integrated circuit (ASIC), a graphics processing unit (GPU), or other integrated circuits (ICs) or may be configured by a combination thereof.

In addition, the video image data input unit 11, the screening unit 12, the recommendation unit 13, the display control unit 14, and the apparatus control unit 17 of the processor 19 can be configured to be partially or entirely integrated into one CPU or the like.

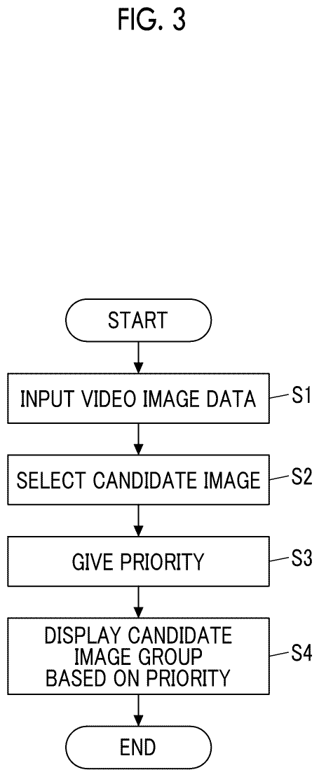

Next, an example of an operation of the image cutout support apparatus according to Embodiment 1 will be described with reference to a flowchart illustrated in FIG. 3. In the following description, it is assumed that the information of the user is input to the image cutout support apparatus in advance.

First, in Step S1, the video image data M composed of the images of a plurality of consecutive frames obtained by imaging the inside of the subject is input to the video image data input unit 11 from an external apparatus, such as an ultrasound diagnostic apparatus (not illustrated), a recording medium (not illustrated), or the like.

Then, in Step S2, the screening unit 12 performs image analysis on the video image data M input in Step S1 to select the candidate image group related to the typical image from the video image data M. For example, the screening unit 12 can extract images for every predetermined number of frames from the video image data M to select the candidate image group. In general, a time interval in a case where images of a plurality of consecutive frames, such as so-called ultrasound images, that represent the anatomical structure of the subject are captured is very short. Therefore, the images of a plurality of frames captured within a certain period of time are often similar to each other. Therefore, in a case where images are extracted for every predetermined number of frames from the video image data M composed of the images of the plurality of consecutive frames, the images similar to each other can be excluded from the object to be selected, and the images having a low degree of similarity can be selected as the candidate image group.

In Step S3, the recommendation unit 13 gives a priority to each image of the candidate image group obtained in Step S2 on the basis of at least one of the image analysis performed by the screening unit 12 or the information of the user. For example, the recommendation unit 13 can give the priority to the image on the basis of at least one of the plurality of conditions, such as the presence or absence of the organ included in the image, the relation between the organ determined by the organ determination and the information of the user, the preference of the user, and the relation between the organ determined by the organ determination and the information of the diagnostic apparatus.

Further, the recommendation unit 13 can also give the priority to the image, using an algorithm called emphasis filtering, in consideration of the plurality of conditions, such as the presence or absence of the organ included in the image, the relation between the organ determined by the organ determination and the information of the user, the preference of the user, and the relation between the organ determined by the organ determination and the information of the diagnostic apparatus.

In this way, the recommendation unit 13 gives a high priority to the image that well represents the anatomical structure or the findings, such as diseases, of the subject, that is, the image that is highly likely to be used, for example, in a case where the user creates a report on the diagnosis of the subject or in a case where the diagnosis result of the subject is shared with other users such as doctors.

Finally, in Step S4, the apparatus control unit 17 displays the candidate image group on the monitor 15 on the basis of the priority given to the candidate image group in Step S3. For example, in a case where the video image data M is composed of ultrasound images of a plurality of frames, the recommendation unit 13 can display the images U1 to U3 of the candidate image group as illustrated in FIG. 2. Therefore, the user can easily ascertain and select the image that well represents the anatomical structure or the findings, such as diseases, of the subject.

In addition, in this case, the apparatus control unit 17 can display the images U4 of a plurality of frames constituting the video image data M input in Step S1. The user can manually select an image from the images U4 of the plurality of frames constituting the video image data M through the input device 18.

In a case where the process in Step S4 is completed in this way, the operation of the image cutout support apparatus according to the flowchart illustrated in FIG. 3 is ended.

As described above, according to the image cutout support apparatus of Embodiment 1 of the present invention, the screening unit 12 performs image analysis on the video image data M to select the candidate image group related to the typical image from the video image data M, and the recommendation unit 13 gives the priority to each image of the candidate image group on the basis of at least one of the image analysis performed by the screening unit 12 or the information of the user. Therefore, the user can easily ascertain and select the image that well represents the anatomical structure or the findings, such as diseases, of the subject.

In addition, the user can set, through the input device 18, the plurality of conditions, such as the presence or absence of the organ included in the image, the relation between the organ determined by the organ determination and the information of the user, the preference of the user, and the relation between the organ determined by the organ determination and the information of the diagnostic apparatus, which are used by the recommendation unit 13 to give the priority to the image in Step S3. For example, in a case where the condition selection buttons B1 to B4 illustrated in FIG. 2 are displayed on the monitor 15 in advance, the user can set the plurality of conditions using the condition selection buttons B1 to B4.

In addition, for example, after the candidate image group is displayed on the basis of the priority in Step S4, it is also possible to change the plurality of conditions used by the recommendation unit 13 to give the priority to the image on the basis of the input operation of the user through the input device 18. In a case where the plurality of conditions have been changed in this way, the process returns to Step S3, and the recommendation unit 13 gives the priority to the candidate image group again under the changed conditions.

### Embodiment 2

An ultrasound diagnostic apparatus can also be formed by adding a configuration of acquiring an ultrasound image to the image cutout support apparatus according to Embodiment 1.

The ultrasound diagnostic apparatus according to Embodiment 2 comprises an ultrasound probe 2 and an apparatus main body 3 connected to the ultrasound probe 2. The ultrasound probe 2 can be connected to the apparatus main body 3 by so-called wired communication or wireless communication.

The ultrasound probe 2 comprises a transducer array 21, and a transmitting and receiving circuit 22 is connected to the transducer array 21.

The apparatus main body 3 comprises an image generation unit 31 connected to the transmitting and receiving circuit 22 of the ultrasound probe 2. In addition, a display control unit 32 and a monitor 33 are sequentially connected to the image generation unit 31. In addition, a video image data input unit 34, a screening unit 35, and a recommendation unit 36 are sequentially connected to the image generation unit 31. Further, the display control unit 32 and an image memory 38 are connected to the recommendation unit 36. In addition, a main body control unit 39 is connected to the transmitting and receiving circuit 22, the image generation unit 31, the display control unit 32, the video image data input unit 34, the screening unit 35, and the recommendation unit 36. Further, an input device 40 is connected to the main body control unit 39.

In addition, a processor 41 for the apparatus main body 3 is configured by the image generation unit 31, the display control unit 32, the video image data input unit 34, the screening unit 35, the recommendation unit 36, and the main body control unit 39. Further, the image cutout support apparatus 42 is configured by the display control unit 32, the monitor 33, the video image data input unit 34, the screening unit 35, the recommendation unit 36, the image memory 38, the main body control unit 39, and the input device 40.

Here, the display control unit 32, the monitor 33, the video image data input unit 34, the screening unit 35, the recommendation unit 36, the image memory 38, and the input device 40 are the same as the display control unit 14, the monitor 15, the video image data input unit 11, the screening unit 12, the recommendation unit 13, the image memory 16, and the input device 18 according to Embodiment 1. Therefore, a detailed description of the display control unit 32, the monitor 33, the video image data input unit 34, the screening unit 35, the recommendation unit 36, the image memory 38, and the input device 40 will be omitted. In addition, the main body control unit 39 is the same as the apparatus control unit 17 according to Embodiment 1 except that the main body control unit 39 controls the transmitting and receiving circuit 22 and the image generation unit 31.

The transducer array 21 of the ultrasound probe 2 includes a plurality of ultrasound transducers which are one-dimensionally or two-dimensionally arranged. Each of the ultrasound transducers transmits ultrasound waves in response to a driving signal supplied from the transmitting and receiving circuit 22, receives ultrasound echoes from the subject, and outputs a signal based on the ultrasound echoes. For example, each of the ultrasound transducers is configured by forming electrodes at both ends of a piezoelectric body consisting of piezoelectric ceramic typified by lead zirconate titanate (PZT), a polymer piezoelectric element typified by polyvinylidene difluoride (PVDF), a piezoelectric single crystal typified by lead magnesium niobate-lead titanate (PMN-PT), or the like.

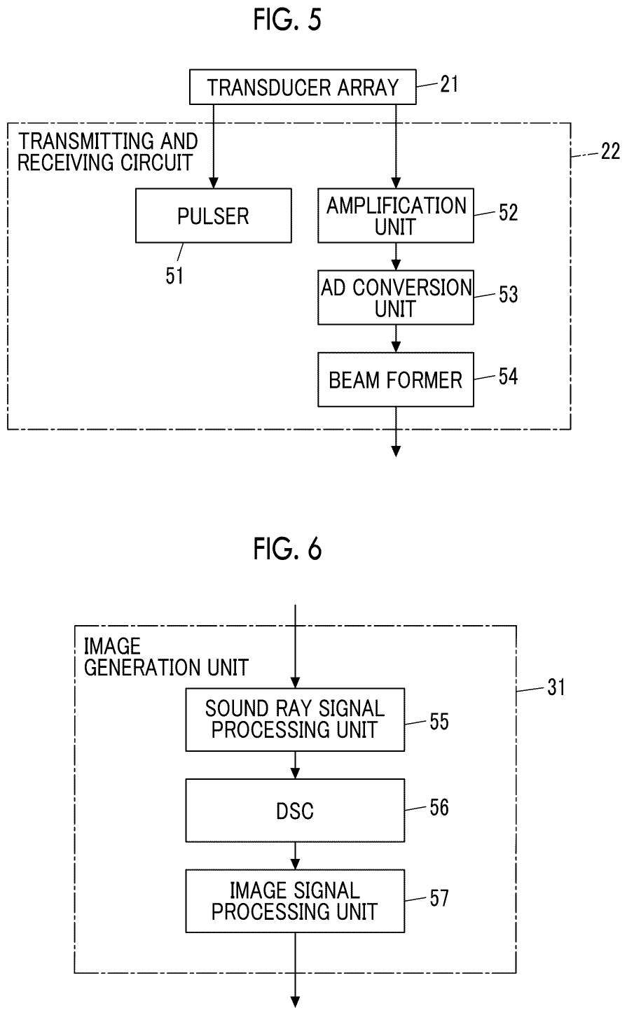

The transmitting and receiving circuit 22 causes the transducer array 21 to transmit the ultrasound waves and generates a sound ray signal on the basis of a received signal acquired by the transducer array 21, under the control of the main body control unit 39. As illustrated in FIG. 5, the transmitting and receiving circuit 22 includes a pulser 51 that is connected to the transducer array 21, and an amplification unit 52, an analog-to-digital (AD) conversion unit 53, and a beam former 54 that are sequentially connected in series to the transducer array 21.

The pulser 51 includes, for example, a plurality of pulse generators, adjusts the amount of delay of each driving signal such that ultrasound waves transmitted from the plurality of ultrasound transducers of the transducer array 21 form an ultrasound beam on the basis of a transmission delay pattern selected according to a control signal from the main body control unit 39, and supplies the driving signal to the plurality of ultrasound transducers. As described above, in a case where a pulsed or continuous-wave voltage is applied to the electrodes of the ultrasound transducers of the transducer array 21, the piezoelectric body is expanded and contracted to generate pulsed or continuous-wave ultrasound waves from each ultrasound transducer, and an ultrasound beam is formed from a combined wave of these ultrasound waves.

The transmitted ultrasound beam is reflected by a target, such as a part of the subject, and propagates toward the transducer array 21 of the ultrasound probe 2. The ultrasound echoes propagating toward the transducer array 21 in this way are received by each ultrasound transducer constituting the transducer array 21. In this case, each of the ultrasound transducers constituting the transducer array 21 receives the propagating ultrasound echoes, is expanded and contracted to generate a received signal which is an electric signal, and outputs the received signal to the amplification unit 52.

The amplification unit 52 amplifies the signal input from each of the ultrasound transducers constituting the transducer array 21 and transmits the amplified signal to the AD conversion unit 53. The AD conversion unit 53 converts the signal transmitted from the amplification unit 52 into digital received data. The beam former 54 applies a delay to each received data item received from the AD conversion unit 53 to perform a so-called reception focus process. Each received data item converted by the AD conversion unit 53 is phase-adjusted and added, and a sound ray signal in which the focus of the ultrasound echo has been narrowed down is acquired by this reception focus process.

As illustrated in FIG. 6, the image generation unit 31 has a configuration in which a sound ray signal processing unit 55, a digital scan converter (DSC) 56, and an image signal processing unit 57 are sequentially connected in series.

The sound ray signal processing unit 55 generates a B-mode image signal, which is tomographic image information related to tissues inside the subject, by performing, on the sound ray signal received from the transmitting and receiving circuit 22, correction of attenuation due to the distance according to the depth of a reflection position of the ultrasound waves using a sound velocity value set by the main body control unit 39 and then performing an envelope detection process on the corrected signal.

The DSC 56 converts (raster-converts) the B-mode image signal generated by the sound ray signal processing unit 55 into an image signal following a normal television signal scanning method.

The image signal processing unit 57 performs various necessary types of image processing, such as gradation processing, on the B-mode image signal input from the DSC 56 to generate an ultrasound image and transmits the generated ultrasound image to the display control unit 32 and the video image data input unit 34. The ultrasound image transmitted to the display control unit 32 is displayed on the monitor 33 through the display control unit 32.

The video image data input unit 34 receives the ultrasound images of a plurality of consecutive frames generated by the image generation unit 31 as the video image data M.

The screening unit 35 performs image analysis on the video image data M input by the video image data input unit 34 to select the candidate image group related to the typical image from the video image data M.

The recommendation unit 36 gives a priority to each image of the candidate image group on the basis of at least one of the image analysis performed by the screening unit 35 or the information of the user.

As described above, according to the ultrasound diagnostic apparatus of Embodiment 2 of the present invention, the screening unit 35 performs the image analysis on the video image data M to select the candidate image group related to the typical image from the video image data M, and the recommendation unit 36 gives the priority to each image of the candidate image group on the basis of at least one of the image analysis performed by the screening unit 35 or the information of the user. Therefore, similarly to the image cutout support apparatus according to Embodiment 1, the user can easily ascertain and select the image that well represents the anatomical structure or findings, such as diseases, of the subject.

In addition, the configuration in which the ultrasound probe 2 comprises the transmitting and receiving circuit 22 has been described. However, instead of the ultrasound probe 2, the apparatus main body 3 may comprise the transmitting and receiving circuit 22.

In addition, the configuration in which the apparatus main body 3 comprises the image generation unit 31 has been described. However, instead of the apparatus main body 3, the ultrasound probe 2 may comprise the image generation unit 31.

### Embodiment 3

Image processing including adjustment of brightness, chroma saturation, and hue can be performed such that the anatomical structure of the subject in the image included in the video image data M is clearly seen.

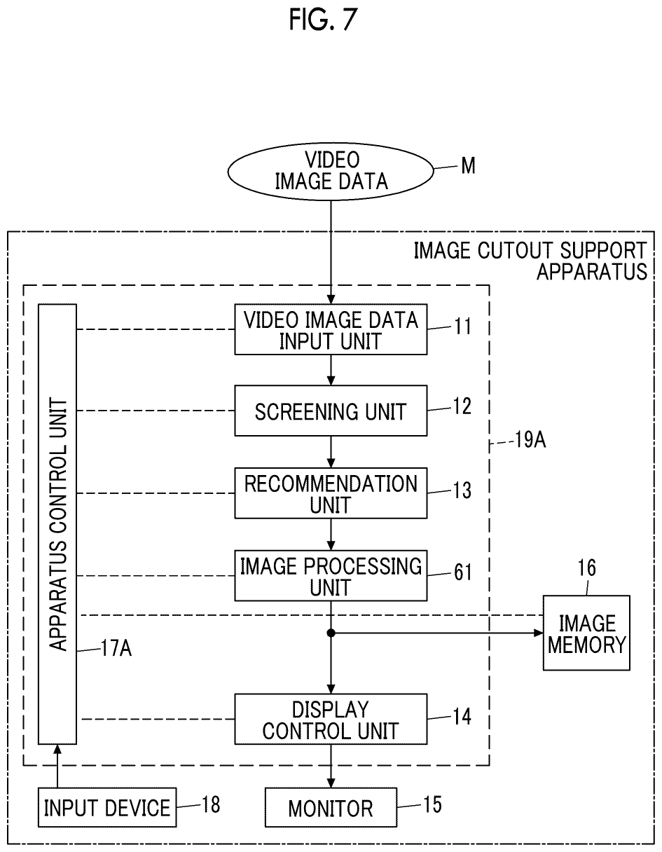

FIG. 7 illustrates a configuration of an image cutout support apparatus according to Embodiment 3. The image cutout support apparatus according to Embodiment 3 differs from the image cutout support apparatus according to Embodiment 1 in that it further comprises an image processing unit 61 and comprises an apparatus control unit 17A instead of the apparatus control unit 17. A processor 19A for the image cutout support apparatus according to Embodiment 3 is configured by the video image data input unit 11, the screening unit 12, the recommendation unit 13, the display control unit 14, the apparatus control unit 17A, and the image processing unit 61.

The image processing unit 61 performs various types of image processing, such as adjustment of brightness, adjustment of chroma saturation, adjustment of hue, and a noise reduction process, on the image included in the video image data M in response to an instruction from the user through the input device 18. For example, the image processing unit 61 can perform the image processing, whose content has been designated by the user, on an image selected by the user through the input device 18 among the images of the plurality of frames constituting the video image data M. This enables the user to obtain, for example, an image obtained by further increasing the sharpness of the image which well represents the anatomical structure or the findings, such as diseases, of the subject and to which a high priority has been given by the recommendation unit 13. The clear image obtained in this way is useful, for example, in a case where the image is published in a report on the diagnosis result of the subject, in a case where the diagnosis result of the subject is shared with other users, such as doctors, and the like.

In addition, the image processing unit 61 can store the content of the image processing performed in the past in response to the instruction of the same user with reference to the information of the user input through the input device 18 and automatically perform the image processing according to the preference of the user on the basis of the stored content of the past image processing. This makes it possible to reduce the time and effort required for the user to input an instruction related to the content of the image processing.

The image subjected to the image processing by the image processing unit 61 is displayed on the monitor 15 through the display control unit 14 and is stored in the image memory 16 under the control of the apparatus control unit 17A.

From the above, according to the image cutout support apparatus of Embodiment 3, the image processing unit 61 performs the image processing on the image included in the video image data M. Therefore, it is possible to acquire an image in which the anatomical structure is clearer.

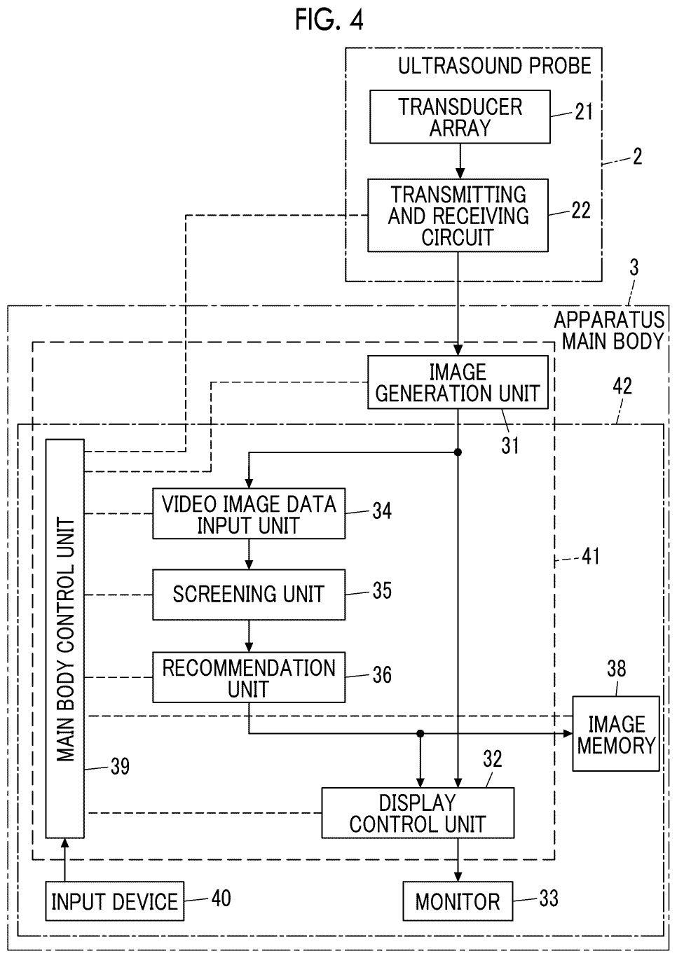

Further, the image processing unit 61 can also be added to the ultrasound diagnostic apparatus according to Embodiment 2 illustrated in FIG. 4. In this case, the image processing unit 61 can perform the image processing on the ultrasound images of the plurality of frames continuously generated by the image generation unit 31. This makes it possible to acquire the ultrasound image in which the anatomical structure is clearer, as in the image cutout support apparatus according to Embodiment 3.

### EXPLANATION OF REFERENCES

2: ultrasound probe
3: apparatus main body
11, 34: video image data input unit
12, 35: screening unit
13, 36: recommendation unit
14, 32: display control unit
15, 33: monitor
16, 38: image memory
17, 17A: apparatus control unit
18, 40: input device
19, 19A, 41: processor
21: transducer array
22: transmitting and receiving circuit
39: main body control unit
42: image cutout support apparatus
51: pulser
52: amplification unit
53: AD conversion unit
54: beam former
55: sound ray signal processing unit
56: DSC
57: image signal processing unit
61: image processing unit
B1 to B4: condition selection button
B5: search button
B6: selection button
M: video image data
N: marker
R1: first display region
R2: second display region
R3: third display region
SB1 to SB3: slide bar
U1 to U4: image
W1: display window

What is claimed is:

1. An image cutout support apparatus for cutting out a typical image including an object to be diagnosed by a user from video image data, the image cutout support apparatus comprising:
a first processor configured to:
input the video image data;
select a candidate image group related to the typical image from the video image data by performing image analysis on the video image data; and
give a priority to each image of the candidate image group based on at least one of the image analysis or information of the user.

2. The image cutout support apparatus according to claim 1,
wherein the first processor is configured to extract images from the video image data for every predetermined number of frames.

3. The image cutout support apparatus according to claim 1,
wherein the first processor is configured to exclude similar images located in neighborhood frames from an object to be selected as the candidate image group.

4. The image cutout support apparatus according to claim 1,
wherein the first processor is configured to exclude an image indicating an aerial emission state from an object to be selected as the candidate image group.

5. The image cutout support apparatus according to claim 1,
wherein the first processor is configured to exclude an image having quality equal to or less than a predetermined threshold value from an object to be selected as the candidate image group.

6. The image cutout support apparatus according to claim 1,
wherein the first processor is configured to:
perform organ determination on each image of the candidate image group; and
give a higher priority to at least one image of the candidate image group including any organ than other images of the candidate image group.

7. The image cutout support apparatus according to claim 2,
wherein the first processor is configured to:
perform organ determination on each image of the candidate image group; and
give a higher priority to at least one image of the candidate image group including any organ than other images of the candidate image group.

8. The image cutout support apparatus according to claim 3,
wherein the first processor is configured to:
perform organ determination on each image of the candidate image group; and
give a higher priority to at least one image of the candidate image group including any organ than other images of the candidate image group.

9. The image cutout support apparatus according to claim 4,
wherein the first processor is configured to:
perform organ determination on each image of the candidate image group; and
give a higher priority to at least one image of the candidate image group including any organ than other images of the candidate image group.

10. The image cutout support apparatus according to claim 5,
wherein the first processor is configured to:
perform organ determination on each image of the candidate image group; and
give a higher priority to at least one image of the candidate image group including any organ than other images of the candidate image group.

11. The image cutout support apparatus according to claim 6,
wherein the first processor is configured to give a further higher priority to an image among the at least one image including an organ which is related to the information of the user.

12. The image cutout support apparatus according to claim 1,
wherein the first processor is configured to give a high priority to at least one image of the candidate image group where a preference of the user has been reflected than other images of the candidate image group, by referring to the information of the user.

13. The image cutout support apparatus according to claim 2,
wherein the first processor is configured to give a high priority to at least one image of the candidate image group where a preference of the user has been reflected than other images of the candidate image group, by referring to the information of the user.

14. The image cutout support apparatus according to claim 3,
wherein the first processor is configured to give a high priority to at least one image of the candidate image group where a preference of the user has been reflected than other images of the candidate image group, by referring to the information of the user.

15. The image cutout support apparatus according to claim 4,
wherein the first processor is configured to give a high priority to at least one image of the candidate image group where a preference of the user has been reflected than other images of the candidate image group, by referring to the information of the user.

16. The image cutout support apparatus according to claim 5,
wherein the first processor is configured to give a high priority to at least one image of the candidate image group where a preference of the user has been reflected than other images of the candidate image group, by referring to the information of the user.

17. The image cutout support apparatus according to claim 1,
wherein the first processor is configured to perform image processing according to a preference of the user based on the information of the user.

18. An ultrasound diagnostic apparatus comprising:
an ultrasound probe;
a second processor configured to transmit and receive an ultrasound beam to and from a subject using the ultrasound probe to generate video image data; and
the image cutout support apparatus according to claim 1,
wherein the video image data generated by the second processor is input to the video image data input unit.

19. An image cutout support method for cutting out a typical image including an object to be diagnosed by a user from video image data, the image cutout support method comprising:
inputting the video image data;
performing image analysis on the video image data to select a candidate image group related to the typical image from the video image data; and
giving a priority to each image of the candidate image group on the basis of at least one of the image analysis or information of the user.
